(19) United States
(12) Patent Application Publication
(10) Pub. No.: US 2020/0326265 A1
(43) Pub. Date: Oct. 15, 2020

(54) MEASUREMENT METHOD OF PIG TWO CELL EMBRYO VOLUME

(71) Applicant: ZhenHua GUO, Herbin (CN)

(72) Inventors: Di LIU, Harbin (CN); ZhenHua GUO, Herbin (CN); Lei LU, Herbin (CN); HongDa WU, Herbin (CN)

(21) Appl. No.: 16/384,395

(22) Filed: Apr. 15, 2019

(51) Int. Cl. G01N 1/30; C12N 5/0735; C12N 5/075; G02B 21/36

(52) U.S. Cl. CPC G01N 1/30; C12N 5/0606; C12N 2509/00; G02B 21/36; C12N 5/0609

(57) ABSTRACT

One measurement method of pig two cell embryo volume. Pig is an important economic animal, the regulation of reproduction can not only provide reference for human reproductive physiology and pathology process research, but also can provide a theoretical basis for improving the reproductive performance of pig. However, the low fertility is a problem plaguing the industry to raise pig. One measurement method of pig two cell embryo volume, based on the figure of pig two cell embryo that was obtained by the microscope, and then measurement, making the regression curve and regression equation. Thus, simple camera and can get more accurate measurement, embryo volume. that is conducive for further research to improve the developmental potential of pig embryo, enhance the production efficiency, is a reliable method to identificatory high quality embryo. The invention is applied to the field of embryo engineering technology.

### FIELD OF THE INVENTION

The invention relates to the invention one measurement method of pig two cell embryo volume.

### BACKGROUND OF THE INVENTION

One measurement method of pig two cell embryo volume. Pig is an important economic animal, the regulation of reproduction can not only provide reference for human reproductive physiology and pathology process research, but also can provide a theoretical basis for improving the reproductive performance of pig. However, the low fertility is a problem plaguing the industry to raise pig. Low fecundity is reflected in all stages, including low meiotic maturation rate, low rate of early embryo development, low pregnancy rate and low live birth rate. Among them, pig and embryo in the early stage of oocyte maturation development process loss has become an important question. Therefore, how to improve the developmental potential of oocytes of pig, to improve the production efficiency, are great significance. In order to improve the success rate of in vitro fertilization (IVF), many number embryo is usually transplanted, but this increases the risk of multiple pregnancies.

In order to improve the success rate, a reliable method of identifying high embryo is needed to be sure for transplantation single eggs. The score system in the past is based on morphological index based, including pronuclear morphology, cleavage rate, blastocyst number and morphology, these indicators are closely related with the activity of embryo. The initial cleavage time of embryo is also a standard for selecting high-quality embryo for transplantation or frozen. Generally, if embryo volume is larger, will be sure have stronger ability of development.

The current measurement method is diameter estimation method

(FIG. 1), but the estimate method of pig two cell embryo volume is not accurate enough, in addition, the same embryo in the rotation of 90 degrees, the observed volume will have a greater change (FIG. 2). Therefore, it is necessary to develop a new and effective method to measure the pig two cell embryo volume, which is helpful to evaluate the developmental potential of pig oocytes cell

### SUMMARY OF THE INVENTION

The purpose of the invention is to provide one measurement method of pig two cell embryo volume.

The purpose of the above is achieved by the following technical scheme:

one measurement method of pig two cell embryo volume, its composition includes: pig two cell embryo, the method consists of the following steps:

(1) Making regression curve

A [symbol] two cell embryo staining, placing polar body in place at 12 clock of embryo, take pictures by microscope, the edges of the measured data are corrected with rectangular frames, and then measured length, get the data of left cell width LX1 [symbol] left cell length LY1 [symbol] right cell width RX1 [symbol] right cell length RY1;

B [symbol] along the direction of the cleavage furrow, embryo is rotated 90 degrees, be sure polar body is up to the embryo, take second photos, the edges of the measured data are corrected with rectangular frames, and then measured length, get the data of left cell width LX2 [symbol] left cell length LY2 [symbol] right cell width RX2 [symbol] right cell length RY2;

C [symbol] LX1 [symbol] LX2 [symbol] LY1 [symbol] LY2 [symbol] RX1 [symbol] RX2 [symbol] RY1 [symbol] RY2 was obtained from step A [symbol] B, then calculated according to the ellipsoid, got the volume of left cell and right cell;

D [symbol] removal of zona pellucida by pronase, separate 2 single cell, take pictures by microscope, the edges of the measured data are corrected with rectangular frames, and then measured length, get the data of left cell width LX [symbol] left cell length LY [symbol] right cell width RX [symbol] right cell length RY;

E [symbol] LX [symbol] RX [symbol] LY [symbol] RY obtained from step D, then calculated according to the sphere, got the volume of left cell and right cell;

F [symbol] volume of left cell and right cell got from step C, that value is considered to be an estimate. volume of left cell and right cell got from step E, that value is considered to be an actual value. 10 embryos were measured according to the above method, regression curve and regression equation were obtained;

(2) measure volume of pig two cell embryo, then estimate volume:

Through the measurement of step A and B, the results are calculated according to regression equation. Pig two cell embryo volume can be obtained without removing the zona pellucida, Embryo development competence can be assessed by continuous culture in vivo.

described measurement method of pig two cell embryo volume, described embryo staining agent is Hoechst 33342.

described measurement method of pig two cell embryo volume, described step A [symbol] B [symbol] D, When taking photos, five consecutive zoom, to ensure that the experiment can get the maximum value of length.

described measurement method of pig two cell embryo volume, described step D, after separation of two cells, If the size of the 2 cell is close, when the zona pellucida is removed, in order to prevent confusion of the left cell and the right cell, a small protrusion is drawn out with holding on the left cell, which can be identified after separation. After 1 hours of recovery in the CO2 incubator, check and turn cell every 20 minutes to prevent cell from growing flat until sphere, take pictures by microscope.

described measurement method of pig two cell embryo volume, described before the zona pellucida is removed, the cell morphology is considered to be close to the ellipsoid. The volume of left cell and right cell are calculated, and the formula is: Measurement volume=(X1+X2)/2×Y1×Y2×⅙×π.

described measurement method of pig two cell embryo volume, described after the zona pellucida is removed, the cell morphology is considered to be close to the sphere. The volume of left cell and right cell are calculated, and the formula is: Measurement volume=⅙×π×[(X+Y)/2]3.

The beneficial effect of the invention:

1. one measurement method of pig two cell embryo volume. Based on the figure of pig two cell embryo that was obtained by the microscope, and then measurement, making the regression curve and regression equation. Thus simple camera and can get more accurate measurement, embryo volume. that is conducive for further research to improve the developmental potential of pig embryo, enhance the production efficiency, is a reliable method to identificatory high quality embryo. The invention is applied to the field of embryo engineering technology.

TABLE 1
10 embryos are measured as follows:

| | | | | | | | | | Measured data after | | | |
|---|---|---|---|---|---|---|---|---|---|---|---|---|
| | Measured data with zona pellucida | | | | | | | | removing zona pellucida | | | |
| No. | LX1 | LY1 | RX1 | RY1 | LX2 | LY2 | RX2 | RY2 | LX | LY | RX | RY |
| 1 | 50.84 | 83.21 | 71.8 | 110.07 | 51.37 | 98.72 | 71.38 | 116.03 | 78.07 | 76.35 | 98.77 | 97.01 |
| 2 | 47.7 | 91.55 | 73.71 | 113.61 | 51.62 | 84.38 | 69.46 | 104.96 | 72.74 | 76.64 | 92.35 | 97.51 |
| 3 | 59.23 | 95.12 | 78.41 | 109.03 | 59.57 | 97.82 | 77.76 | 112.95 | 79.59 | 86.22 | 100.2 | 104.2 |
| 4 | 56.16 | 96.09 | 64.31 | 97.97 | 55.81 | 92.63 | 58.38 | 90.07 | 83.4 | 80.22 | 83.03 | 86.26 |
| 5 | 58.11 | 94.18 | 67.95 | 106.26 | 59.27 | 99.48 | 65.62 | 107.57 | 85.91 | 83.51 | 94.02 | 95 |
| 6 | 53.77 | 92.62 | 67.26 | 103.77 | 53.32 | 91.15 | 66.63 | 105.18 | 76.5 | 81.83 | 89.94 | 92.22 |
| 7 | 53.73 | 92.58 | 71.71 | 103.64 | 54.25 | 90.52 | 69.24 | 103.6 | 79.33 | 76.82 | 92.28 | 95.32 |
| 8 | 40.74 | 73.38 | 81.3 | 119.28 | 36.67 | 82.67 | 87.12 | 118.31 | 62.98 | 57.06 | 103.21 | 111 |
| 9 | 51.31 | 86.84 | 72.05 | 105.17 | 53.64 | 86.65 | 70.86 | 103.11 | 78.16 | 73.68 | 96.52 | 91.32 |
| 10 | 59.44 | 97.75 | 61.81 | 98.61 | 56.81 | 97.67 | 63.68 | 100.56 | 84.42 | 81.83 | 84.93 | 89.22 |

TABLE 2
10 embryos are calculated and the following data are obtained:

| | |
|---|---|
| Measured data with | Measured data after |
| zona pellucida | removing zona pellucida |

| | left cell | right cell | left cell | right cell |
|---|---|---|---|---|
| No. | volume | volume | volume | volume |
| 1 | 219.70 | 478.49 | 240.85 | 490.86 |
| 2 | 200.76 | 446.73 | 217.91 | 447.37 |
| 3 | 289.24 | 503.24 | 297.73 | 558.59 |
| 4 | 260.78 | 283.29 | 286.44 | 317.27 |
| 5 | 287.76 | 399.50 | 318.05 | 441.77 |
| 6 | 236.57 | 382.39 | 259.35 | 395.35 |
| 7 | 236.78 | 396.00 | 249.00 | 431.79 |
| 8 | 122.88 | 621.92 | 112.88 | 641.71 |
| 9 | 206.64 | 405.51 | 228.81 | 433.23 |
| 10 | 290.42 | 325.61 | 300.52 | 345.30 |

The linear regression is obtained of the 20 data, data with the zona pellucida (estimated value) contrast to the removed zona pellucida (actual value). Result as shown in FIG. 8. The fitting degree of R²=0.9885, and the regression equation is obtained: Y=1.04X+7.67. Y is actual value measured from the embryo removing the zona pellucida, and the X is calculated according to the two cell embryo measurement results before removing the zona pellucida.

G According to the regression curve, through the step A, B measurement FIG. 9 embryo, using regression equation: Y=1.04X+7.67 calculations of volume. You can get it without removing the zona pellucida two cell embryo volume left=269 μm³, right=418 μm³, Embryo development competence can be assessed by continuous culture in vivo.

This example still removes the zona pellucida, separation of 2 cell, that has been measured in FIG. 10, calculated the actual volume left=286 μm³, 1.06 times of estimated value, right=433 μm³, 0.98 times of estimation value, in order to be sure, the reliability of the present invention.

From the above test data, it can be known that the error value of pig two cell embryo volume estimated by the method of the invention can be controlled within 10%.

### BRIEF DESCRIPTION OF THE DRAWINGS

FIG. 1 is traditional measurement method of pig two cell embryo volume: schematic diagram of diameter estimation method.

FIG. 2A and FIG. 2B is the schematic diagram of change angle after the same embryo rotation 90 degrees.

FIG. 2A is the polar body at 12 o'clock position; FIG. 2B is the position of the polar body after 90 degrees of rotation.

FIG. 3A is the schematic diagram of pig two cell embryo volume before removing the zona pellucida. The same embryo measurement is obtained left cell width LX1, left cell length LY1, right cell width RX1, and right cell length RY1.

FIG. 3B is the schematic diagram of pig two cell embryo volume when embryo is rotated 90 degrees along the direction of the cleavage furrow. The edges of the measured data are corrected with rectangular frames, and then measured length, get the data of left cell width LX2, left cell length LY2, right cell width RX2, and right cell length RY2.

FIG. 4A and FIG. 4B is the schematic diagram of the 3D simulation of the position change of the polar body after the same embryo rotation 90 degrees.

FIG. 4A is the polar body at 12 o'clock position; and FIG. 4B is the polar body rotated 90 degrees, above the embryo.

FIG. 5A and FIG. 5B is the schematic diagram of cell after removing 2 cell from the zona pellucida.

FIG. 6A and FIG. 6B is a schematic diagram to prevent confusion when removing the transparent belt. In order to prevent the confusion of the left cell and right cell. Used a needle in the left cell a little hard to suck a small bump as shown in FIG. 6A. After the separation can be identified as shown in FIG. 6B.

FIG. 7A and FIG. 7B is pig two cell embryo, after cell separation diagram was measured in which FIG. 7A gets the data of left cell width LX, left cell length LY, and FIG. 7B gets the data of right cell width RX, right cell length RY.

FIG. 8 is the schematic diagram of the regression curve in example 1. Y is actual value measured from the embryo removing the zona pellucida, and the X is calculated according to the two cell embryo measurement results before removing the zona pellucida.

FIG. 9 is example 1, pig two cell embryo diagram before removing the zona pellucida.

FIG. 10: Example 1, pig two cell embryo, cell separation volume measurement example diagram.

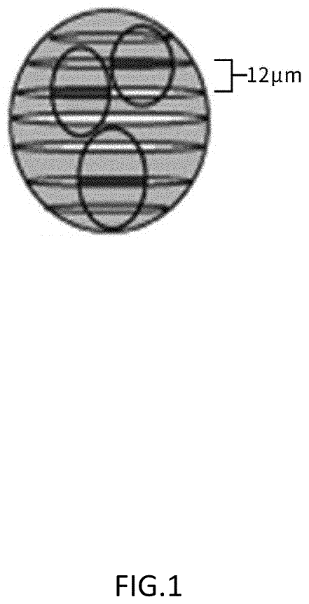

### DESCRIPTION OF THE PREFERRED EMBODIMENTS

### Example 1

one measurement method of pig two cell embryo volume, its composition includes: pig two cell embryo, the method consists of the following steps:

(1) Making regression curve

A [symbol] two cell embryo staining, placing polar body in place at 12 clock of embryo, take pictures by microscope, the edges of the measured data are corrected with rectangular frames, and then measured length, get the data of left cell width LX1 [symbol] left cell length LY1 [symbol] right cell width RX1 [symbol] right cell length RY1;

B [symbol] along the direction of the cleavage furrow, embryo is rotated 90 degrees, be sure polar body is up to the embryo, take second photos, the edges of the measured data are corrected with rectangular frames, and then measured length, get the data of left cell width LX2 [symbol] left cell length LY2 [symbol] right cell width RX2 [symbol] right cell length RY2;

C [symbol] LX1 [symbol] LX2 [symbol] LY1 [symbol] LY2 [symbol] RX1 [symbol] RX2 [symbol] RY1 [symbol] RY2 was obtained from step A [symbol] B, then calculated according to the ellipsoid, got the volume of left cell and right cell;

D [symbol] removal of zona pellucida by pronase, separate 2 single cell, take pictures by microscope, the edges of the measured data are corrected with rectangular frames, and then measured length, get the data of left cell width LX [symbol] left cell length LY [symbol] right cell width RX [symbol] right cell length RY;

E [symbol] LX [symbol] RX [symbol] LY [symbol] RY obtained from step D, then calculated according to the sphere, got the volume of left cell and right cell;

F [symbol] volume of left cell and right cell got from step C, that value is considered to be an estimate. volume of left cell and right cell got from step E, that value is considered to be an actual value. 10 embryos were measured according to the above method, regression curve and regression equation were obtained;

(2) measure volume of pig two cell embryo, then estimate volume:

Through the measurement of step A and B, the results are calculated according to regression equation. Pig two cell embryo volume can be obtained without removing the zona pellucida, Embryo development competence can be assessed by continuous culture in vivo.

### Example 2

According to embodiment 1 described measurement method of pig two cell embryo volume, described embryo staining agent is Hoechst 33342.

### Example 3

According to embodiment 1 or 2 described measurement method of pig two cell embryo volume, described step A [symbol] B [symbol] D, When taking photos, five consecutive zoom, to ensure that the experiment can get the maximum value of length.

### Example 4

According to embodiment 1 or 2 or 3 described measurement methods of pig two cell embryo volume, described step D, after separation of two cells, If the size of the 2 cell is close, when the zona pellucida is removed, in order to prevent confusion of the left cell and the right cell, a small protrusion is drawn out with holding on the left cell, which can be identified after separation. After 1 hours of recovery in the CO2 incubator, check and turn cell every 20 minutes to prevent cell from growing flat until sphere, take pictures by microscope.

### Example 5

According to embodiment 1 or 2 or 3 or 4 described measurement methods of pig two cell embryo volume, described before the zona pellucida is removed, the cell morphology is considered to be close to the ellipsoid. The volume of left cell and right cell are calculated, and the formula is: Measurement volume=(X1+X2)/2×Y1×Y2×⅙×π.

### Example 6

According to embodiment 1 or 2 or 3 or 4 or 5 described measurement method of pig two cell embryo volume, described after the zona pellucida is removed, the cell morphology is considered to be close to the sphere. The volume of left cell and right cell are calculated, and the formula is: Measurement volume=⅙×π×[(X+Y)/2]3.

### Example 7

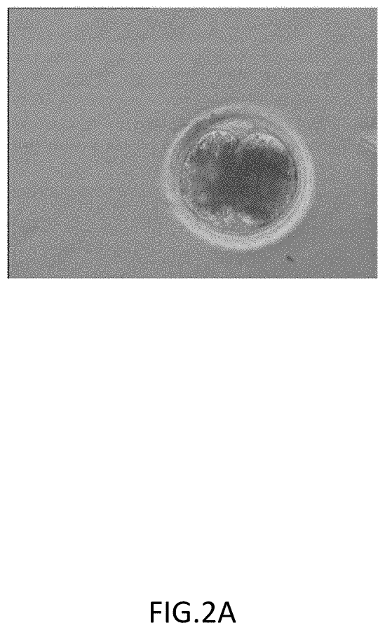

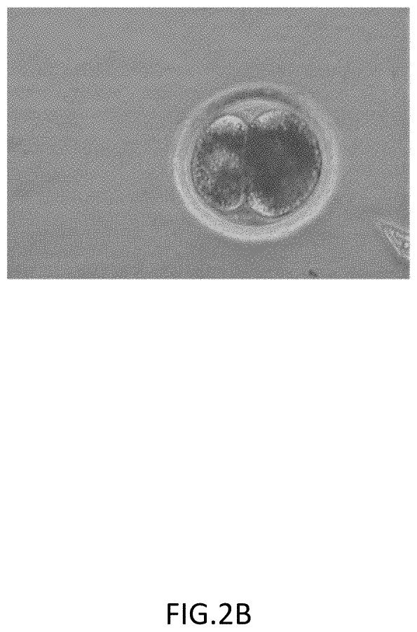

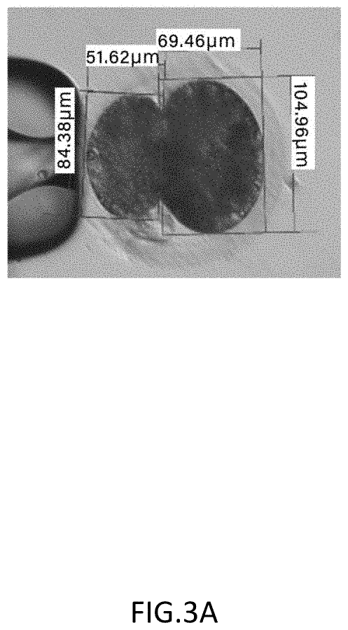

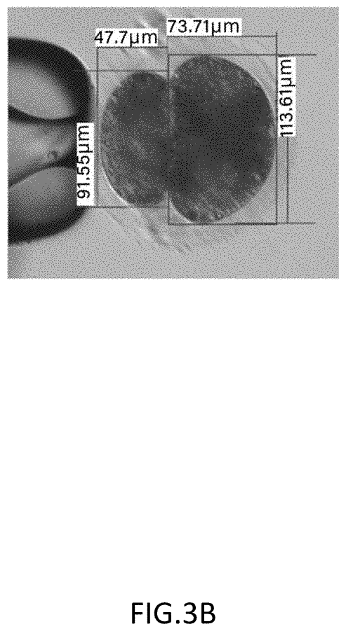

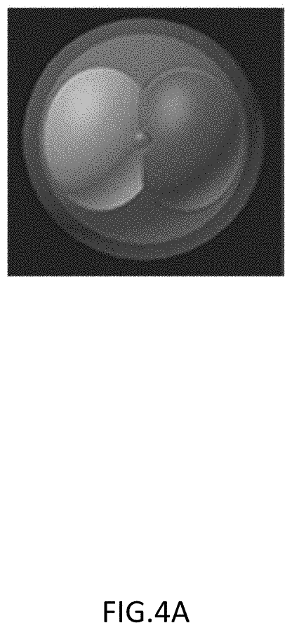

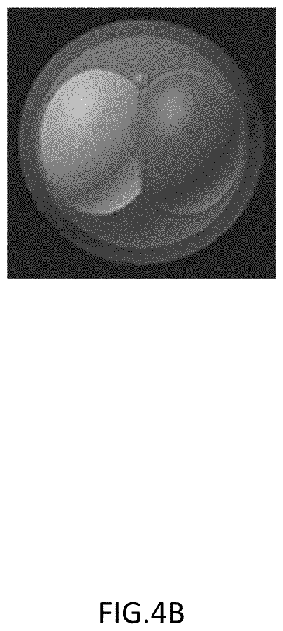

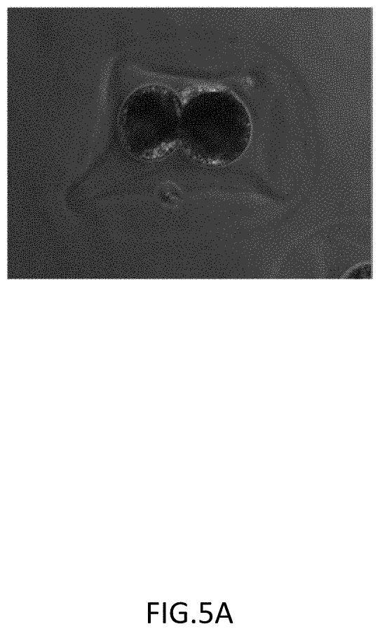

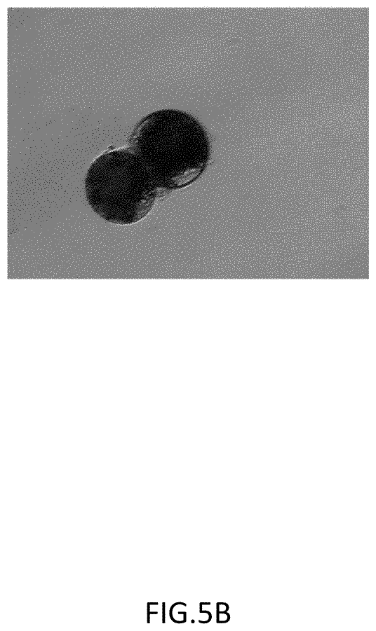

As shown in FIG. 2 of the manual, the same embryo will lead to different data acquisition. Embryo left photos rotate 90 degrees to get the right photos, so you need to take first photos and rotate 90 degrees take second photos.

### Example 8

According to the manual 3, the same embryo measurement is obtained left cell width LX1 [symbol] left cell length LY1 [symbol] right cell width RX1 [symbol] right cell length RY1; along the direction of the cleavage furrow, embryo is rotated 90 degrees, take second photos, the edges of the measured data are corrected with rectangular frames, and then measured length, get the data of left cell width LX2 [symbol] left cell length LY2 [symbol] right cell width RX2 [symbol] right cell length RY2.

### Example 9

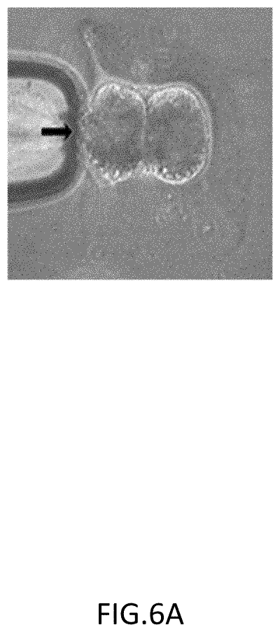

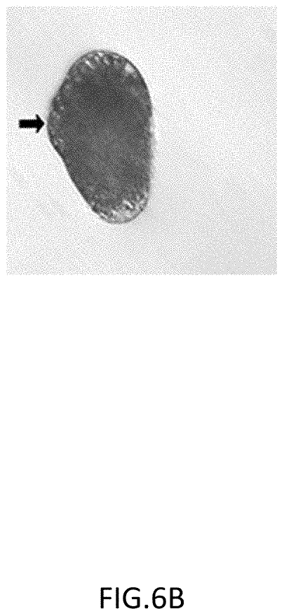

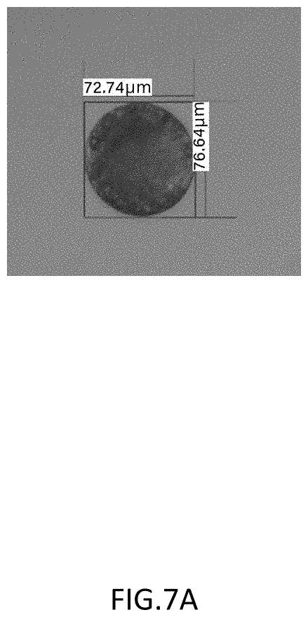

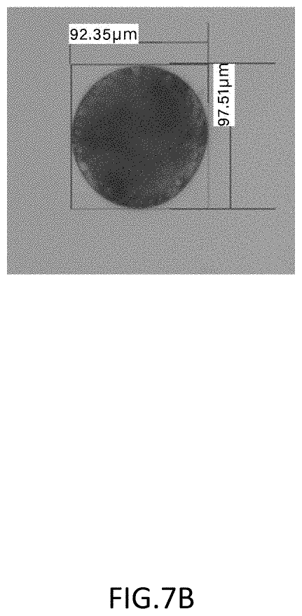

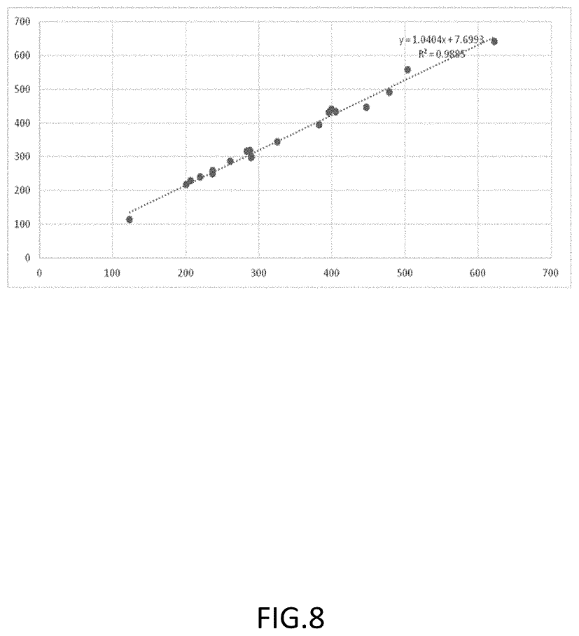

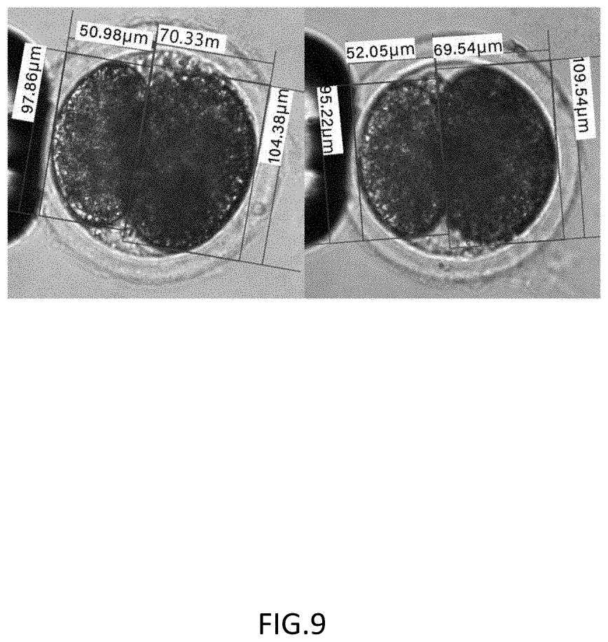

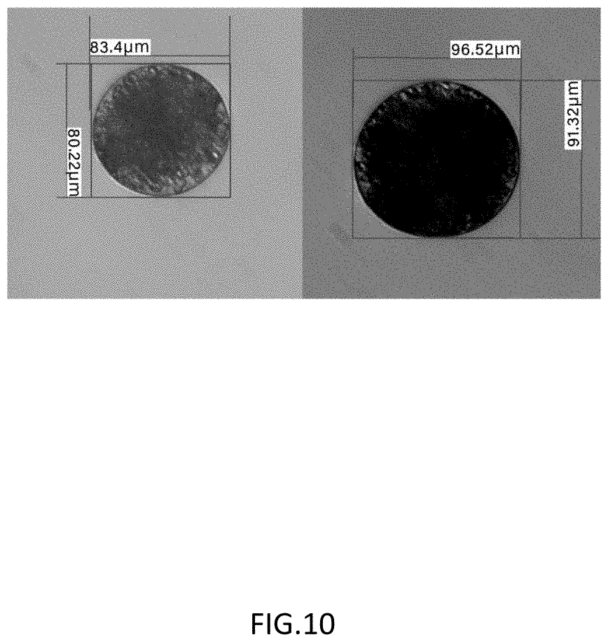

According to FIG. 6 shows, when the 2 single cell size is close, in order to prevent the confusion of the left cell and right cell. Used a needle in the left cell a little hard to suck a small bump as shown in A, after the separation can be identified as shown in B.

### Example 10

According to the manual 9, the same embryo measurement is obtained left cell width LX1 [symbol] left cell length LY1 [symbol] right cell width RX1 [symbol] right cell length RY1; along the direction of the cleavage furrow, embryo is rotated 90 degrees, take second photos, the edges of the measured data are corrected with rectangular frames, and then measured length, get the data of left cell width LX2 [symbol] left cell length LY2 [symbol] right cell width RX2 [symbol] right cell length RY2.

What is claimed is:

1. one measurement method of pig two cell embryo volume, its composition includes: pig two cell embryo, its characteristic is that: the method consists of the following steps:
(1) Making regression curve
A [symbol] two cell embryo staining, placing polar body in place at 12 clock of embryo, take pictures by microscope, the edges of the measured data are corrected with rectangular frames, and then measured length, get the data of left cell width LX1 [symbol] left cell length LY1 [symbol] right cell width RX1 [symbol] right cell length RY1;
B [symbol] along the direction of the cleavage furrow, embryo is rotated 90 degrees, be sure polar body is up to the embryo, take second photos, the edges of the measured data are corrected with rectangular frames, and then measured length, get the data of left cell width LX2 [symbol] left cell length LY2 [symbol] right cell width RX2 [symbol] right cell length RY2;
C [symbol] LX1 [symbol] LX2 [symbol] LY1 [symbol] LY2 [symbol] RX1 [symbol] RX2 [symbol] RY1 [symbol] RY2 was obtained from step A [symbol] B, then calculated according to the ellipsoid, got the volume of left cell and right cell;
D [symbol] removal of zona pellucida by pronase, separate two cell embryo, take pictures by microscope, the edges of the measured data are corrected with rectangular frames, and then measured length, get the data of left cell width LX [symbol] left cell length LY [symbol] right cell width RX [symbol] right cell length RY;
E [symbol] LX [symbol] RX [symbol] LY [symbol] RY obtained from step D, then calculated according to the sphere, got the volume of left cell and right cell;
F [symbol] volume of left cell and right cell got from step C, that value is considered to be an estimate. volume of left cell and right cell got from step E, that value is considered to be an actual value. 10 embryos were measured according to the above method, regression curve and regression equation were obtained;
(2) measure volume of pig two cell embryo, then estimate volume:
Through the measurement of step A and B, the results are calculated according to regression equation. Pig two cell embryo volume can be obtained without removing the zona pellucida, Embryo development competence can be assessed by continuous culture in vivo.

2. according to the claim 1 described measurement method of pig two cell embryo volume, its characteristic is that: described embryo staining agent is Hoechst 33342.

3. according to the claim 1 described measurement method of pig two cell embryo volume, its characteristic is that: described step A [symbol] B [symbol] D, When taking photos, five consecutive zoom, to ensure that the experiment can get the maximum value of length;
the step D, after separation of two cells, If the size of the 2 cell is close, when the zona pellucida is removed, in order to prevent confusion of the left cell and the right cell, a small protrusion is drawn out with holding on the left cell, which can be identified after separation. After 1 hours of recovery in the CO2 incubator, check and turn cell every 20 minutes to prevent cell from growing flat until sphere, take pictures by microscope;
the zona pellucida is removed, the cell morphology is considered to be close to the ellipsoid. The volume of left cell and right cell are calculated, and the formula is: Measurement volume=(X1+X2)/2×Y1×Y2×⅙×π; and
the zona pellucida is removed, the cell morphology is considered to be close to the sphere. The volume of left cell and right cell are calculated, and the formula is: Measurement volume=⅙×π×[(X+Y)/2]3.

4. according to the claim 2 described measurement method of pig two cell embryo volume, its characteristic is that: described step A [symbol] B [symbol] D, When taking photos, five consecutive zoom, to ensure that the experiment can get the maximum value of length;
The step D, after separation of two cells, If the size of the 2 cell is close, when the zona pellucida is removed, in order to prevent confusion of the left cell and the right cell, a small protrusion is drawn out with holding on the left cell, which can be identified after separation. After 1 hours of recovery in the CO2 incubator, check and turn cell every 20 minutes to prevent cell from growing flat until sphere, take pictures by microscope;
the zona pellucida is removed, the cell morphology is considered to be close to the ellipsoid. The volume of left cell and right cell are calculated, and the formula is: Measurement volume=(X1+X2)/2×Y1×Y2×⅙×π; and;
the zona pellucida is removed, the cell morphology is considered to be close to the sphere. The volume of left cell and right cell are calculated, and the formula is: Measurement volume=⅙×π×[(X+Y)/2]3.
